(19) United States
(12) Patent Application Publication
(10) Pub. No.: US 2022/0346869 A1
(43) Pub. Date: Nov. 3, 2022

(54) MEDICAL IMPLANTABLE INTERATRIAL SEPTAL DEFECT OCCLUSION DEVICE

(71) Applicant: SQUADRA Lifesciences, Inc., Palo Alto, CA (US)

(72) Inventors: EUSTAQUIO MARIA ONORATO, Gussago (IT); ANTONIO COLOMBO, Milan (IT); MEHMET HAKAN AKPINAR, Istanbul (TR)

(73) Assignee: SQUADRA Lifesciences, Inc., Palo Alto, CA (US)

(21) Appl. No.: 17/858,802

(22) Filed: Jul. 6, 2022

Related U.S. Application Data
(63) Continuation of application No. PCT/US21/13312, filed on Jan. 13, 2021.
(60) Provisional application No. 62/960,989, filed on Jan. 14, 2020.

(51) Int. Cl. A61B 18/14

(52) U.S. Cl. CPC A61B 18/1492; A61B 2017/00867

(57) ABSTRACT

The present invention relates to a medical implantable interatrial septal defect occlusion device to occlude the congenital cardiac malformations such as Atrial Septal Defect (ASD) and Patent Foramen Ovale (PFO) providing hemodynamics between two atria. The occlusion device includes distal and proximal discs having expandable shape memory characteristics, pre-created sealed potential fenestrations which are sealed with biocompatible polymeric patch and sutures to be perforated and used for any possible intervention needed.

### CROSS-REFERENCE TO RELATED APPLICATIONS

This application is a continuation of PCT Application No. PCT/US2021/013312 (Attorney Docket No. 55631-704.601, filed Jan. 13, 2021, which claims the benefit of U.S. Provisional No. 62/960,989 (Attorney Docket No. 55631-704.101), filed on Jan. 14, 2020, the entire content of which is incorporated herein by reference.

### BACKGROUND OF THE INVENTION

### Field of the Invention

This invention relates to a medical implantable interatrial septal defect occlusion device which includes distal and proximal discs having expandable shape memory characteristics and braided mesh with biocompatible polymeric patch and sutures to occlude the congenital cardiac malformations such as Atrial Septal Defect (ASD) and Patent Foramen Ovale (PFO) providing hemodynamics between two atria. More particularly, the device contains pre-created potential fenestrations which are sealed with biocompatible polymeric patch and sutures on discs to be opened/perforated and used for any possible intervention needed.

Atrial Septal Defect (ASD) and Patent Foramen Ovale (PFO) are types of congenital cardiac defects causing abnormal pathways for harmful hemodynamics between two atria. The Atrial Septal Defect (ASD) is one of the most common types of congenital heart disease that allow communication between the left and right sides of the heart. These interatrial communications include several distinct defects in the cardiac terminations of the systemic and pulmonary veins (sinus venous and coronary sinus defects) and the interatrial septum (atrial septal defects). PFO is a normal communication during fetal life and is commonly encountered after birth. PFO can be identified in high rates of the population by echocardiography but some of them survive all their life without any treatment. However, some of them have experienced times of strokes or transient ischemic attacks that are related to damaged heart atrial function. Transcatheter closure of a PFO reduces the risk of recurrent cryptogenic stroke compared with medical therapy. The people with an ASD or PFO may suffer from complications, including peripheral embolism, thrombosis, and arterial hypertension.

Device closure of congenital heart diseases through the transcatheter approach has now been well accepted as an option for surgical treatment. Currently, there are many kinds of occlusion devices available in the market. It has been obtained a high rate of success using occluder devices for ASD and PFO but still, some problems such as residual shunt occur related to the type of occluder and morphology of defect. Complete closure depends on the growth of the endothelium to cover the device and the septum, known as endothelialization. Therefore, geometrical compatibility between the device and the defect is important for the endothelization process.

Thrombotic events pose a great threat to patients with PFO and ASD, which can be effectively prevented by percutaneous closure, as reported in many previous studies. Percutaneous closure can also improve the symptoms of migraine. It is suggested that PFO has a higher thrombus formation rate due to its higher residual shunt rate and slower blood flow interatrial.

Early in 1974, King and Mills reported for the first time the transcatheter closure of ASD using a double-umbrella device. Rashkind and Mullins developed the first commercially available device in the early 1980s, namely the Rashkind device. Later on, many devices have been developed to design a reliable and safe closure system. Closure devices for ASD can also be used for preventing paradoxical embolism in transcatheter closure of PFO.

Currently, there are several types of occlusion devices for ASD and PFO. The available occlusion devices in the market have a metallic frame that consists of one or multiple wires, a polymeric patch covering the metallic frame, and sutures for fixing the patches. Most of the devices have the same double-disc concept in the market such as Amplatzer Cribriform occluder, Amplatzer ASD occluder, Amplatzer PFO, Occlutech occluders, CARDIOFORM Septal Occluder but also there are different concepts like spiral shape single strand nitinol wire covered with a thin mesh of expandable polytetrafluoroethylene (PTFE) patch (Helex Septal Occluder) or two self-expanding square umbrellas made of polyethylene terephthalate (PET) patches (CardioSEAL or CardioSEAL-STARFlex devices). The Amplatzer occluders are the most common occluders for transcatheter closure of ASD and PFO defects but Tang et al. have reported that they have some disadvantages like thrombus formation and minor complications. Tang et al. indicated that the Occlutech occluders have some disadvantages like residual shunt and less published experienced. Residual leak and fracture of the metallic wireframe are reported by the same group as complications using Helex Septal Occluder.

In the prior art, discs of PFO device have stretched both sides because of the design of the waist in between discs, where they have a convex or concaved shape which creates a residual shunt. Prior art devices have a single-point connection between the discs which creates a risk of residual shunt because the waist area permits the blood flow after implantation and does not occlude the defect area completely. Also, devices in the prior art have two cross-shaped nitinol anchors that one-side of the anchor is covered by double layers of knitted PET fabric (Premere PFO occluder) or a shape memory metallic skeleton, eight wires, and two PET patches fixed by two-wire holders (Solysafe Septal Occluder). The residual shunt is a common complication related to these occluders. Moreover, in the prior art, some devices comprises a double umbrella made of polyvinyl alcohol (PVA) and six-stranded nitinol wire arms and an additional foam plug is present between the two umbrellas (Intrasept). Luermans et al. have reported some complications related to the Intrasept device such as cryptogenic stroke, transient ischemic attack, peripheral embolism. Another device has also nitinol wire mesh and two left atrial anchors that are directly inserted into the PFO pocket (SeptRx) but Tang et al. indicated that there is less experience with the device.

In the prior art, there is also a novel suture-based “deviceless” Noble Stitch EL system is available. This device consists of 2 polypropylene sutures, 1 for the septum primum and 1 for the septum secundum, tightened together by a dedicated delivery and sealing system. The defect is closed and fixed by just applying a suture during operation with this system, and then the delivery device is taken out from the body. This method can be performed only in suitable anatomies of defects.

Transcatheter closure treatment with closure devices presents many advantages including safety, ease of operation, minimal invasiveness, and few complications. However, there is a problem in the prior art which is insufficient closure of the defect by occlusion devices due to the device geometry. The occlusion device can not cover the defect geometry completely, especially in patent foramen ovale defects due to defect geometry has not in a common shape and can lead to residual shunt. Another problem is that previous occlusion devices require a high level of precision and skill, longer time with a potential risk of insufficient closure of the defect. Moreover, the present occluder devices in the prior art do not allow physicians to intervene if there is a need to cross the interatrial septum post-implantation. Some physicians perforate the occluders when there is an intervention needed during operations and they need different calibrations of fenestration for different patients. When a certain diameter of fenestration is needed, occluders with fenestration do not meet the requirement of the physicians since the fenestration is not appropriate for every operation. Thus, these problems may lead to a high risk for the patient and the health care system.

According to the problems in the prior art, an occlusion device having a geometry that is more compatible with the cardiac defect to avoid complications like residual shunt related to insufficient closure of congenital cardiac defects and allowing access to both sides of the atrium when an interventional trans-septal procedure is needed in this technical field.

### SUMMARY OF THE INVENTION

This invention relates to a medical implantable interatrial septal defect occlusion device which includes distal and proximal discs having expandable shape memory characteristics and braided mesh with biocompatible polymeric patch and sutures to occlude the congenital cardiac malformations such as Atrial Septal Defect (ASD) and Patent Foramen Ovale (PFO) providing hemodynamics between two atria. More particularly, the device contains pre-created potential fenestrations which are sealed with biocompatible polymeric patch and sutures on discs to be opened/perforated and used for any possible intervention needed.

The aim of the invention is the development of an occlusion device having a geometry that is more compatible with the cardiac defect geometry and elimination of complications resulted from insufficient closure of congenital cardiac defects. The invention comprises left atrial disc, right atrial disc, flat connecting waist, and angulation between two discs to provide excellent fitting for congenital cardiac malformations and minimizes the risk of shunt caused by undersizing or oversizing that may lead to the forming of non-occluded area.

Another aim of the invention is to create a fusion between the device surface and the tissues of the interatrial septal defect, septal walls. To provide the fusion and perfect attachment of the device to the tissues, the device contains electrodes connected to the surrounding metallic braided mesh discs which are in contact with the tissues of the interatrial septal defect to transfer energy like radio frequency (RF), heat, or similar, to create a fusion between the device surface and the tissue. The pusher cable for this device also has a core section with isolated conductive metallic wires which are used to deliver the energy to the device after the implantation.

Another aim of the invention is to allow physicians to access both sides of the atrium when an interventional trans-septal procedure is needed. The sealed fenestrations with biocompatible polymeric patch and sutures on discs perfectly close the septal defects in the septum. In the present invention, there is not an exact fenestration in open-form, there are pre-creations of the fenestrated frames in the structure of the metallic braided mesh to be perforated to achieve either access to the other ventricular side or blood perfusion to change or regulate the pressure gradient between two atriums to perform a medical therapy to the patient after implantation of the device acute or chronically. These fenestrations on the mesh structure are sealed or covered with patches so that they are in closed or sealed form. And these sealed fenestrations act as potential fenestrations that can be perforated later if an interventional trans-septal operation is needed.

Another purpose of the invention is to prevent the stretching out of the discs of the occluder and provide a better seal. The connecting waist of the braided pre-shaped metallic structure of the present invention has a flat geometrical design and a certain angulation between the discs and waist that has closure and seal properties within the defect location, independent from the closure of the discs to seal both sides of the defect. The design of the connecting waist in the present invention eliminates the effect of stretching out the discs and close the defect tunnel and prevent residual shunt. On the contrary to the prior art, the present invention provides a flat connection between discs so that the defect area is closed completely by the connecting waist and eliminates the risk of the residual shunt.

The present invention overcomes the problems which are insufficient closure of the interatrial septal defect, not having a compatible geometry with the cardiac defect, not allowed to intervene to cross the interatrial septum post-implantation, and other attachment disadvantages present in the prior art by providing a device having a geometry that is more compatible with the cardiac defect to avoid complications like residual shunt, having electrodes connected to the surrounding nitinol mesh discs to transfer energy to the device and tissue to fuse them for a perfect attachment and having pre-created sealed potential fenestrations to allow access to either side of the atrium when an interventional trans-septal procedure is needed.

In a fisrt aspect, the present invention comprses a device for occluding an interatrial septal defect, such as a patent a patent foramen ovale (PFO) or an atrial septal defect (ASD). The device comprises an expandable frame structure formed from a nickel-titanium alloy or other shape-memory metal mesh (105) and having a left atrial disc (101), a right atrial disc (106), and a waist (107, 112, 117) connecting the left atrial disc (101) and the right atrial disc (106). At least one fenestration (102) is located on the left atrial disc (101) and at least one fenestration (102) located on the right atrial disc (106). A biocompatible polymeric patch (111) is located both on the left atrial disc (101) to seal the at least one fenestration (102) and on the right atrial disc (106) to seal the at least one fenestration (102), wherin said biocompatible polymeric patch is configured to be perforated to allow access therethrough when needed.

In particular embodiments, the device further may comprise at least one radiopaque marker (108) located on the left and/or the right biocompatible polymeric patch (111) to indicate a location of one or more of the fenestrations (102). The device may also further comprise a connecting hub (116) configured to be attached to a pusher cable (109) which contains electrodes (113) to transfer energy to the device surface and septum tissue for fusion.

In any such devices, the waist (107, 112, 117) may be in a flat form in between the left atrial disc (101) and the right atrial disc (106) or may be in a cylindrical form in between the left atrial disc (101) and the right atrial disc (106).

In any such devices, the biocompatible polymeric patch (111)may comprise a material selected from the group consisting of polytetrafluoroethylene (PTFE), polyethylene terephthalate (PET), a polyester (Dacron®), apolyurethane (PU), or a bioabsorbable polymeric material.

In some instances, the device may comprise a plurality of fenestrations (102) located on at least one of the left atrial disc (101) and the right atrial disc (106) of the metallic braided mesh (105).

In some instances, at least some of the plurality of fenestrations (102) may have different sizes from each other, while in other instnce the sizes for some or all will be identical.

The devices of the presnt invention may comprise one layer of the biocompatible polymeric patch (111) on the left atrial disc (101) and a separate layer of the biocompatible polymeric patch (111) located on the right atrial disc (106) to provide hemostatic sealing. For example, there may be three layers of the biocompatible polymeric patch (111) on metallic braided mesh (105) including a first layer is located on the left atrial disc (101), a second layer is located on the right atrial disc (106), and a third layer is located in the waist to provide hemostatic sealing.

In any such devices, the left atrial disc (101) and right atrial disc (106) may be formed as full circles. Alternativley, the left atrial disc (101) and right atrial disc (106) are formed as half circles.

In any such devices, the waist may be configured to allow an angulation bwtween the left atrial disc (101) and the right atrial disc (106) in a range from 15°to 90°.

In any such devices, the radiopaque marker (108) is located on the patch (111) of the right atrial disc (106) only.

In any such devices, the metallic braided mesh (105) may be made of a shape memory metal alloy which is superelastic. wherein said metal alloy comprises a nickel-titanium alloy (Nitinol®).

In any such devices, the shape-memory metal mesh may comprise in whole or in part a fully braided structure. Alternatively, the shape-memory metal mesh may comprise in whole or in part a partially braided structure. Still further alternatively, the shape-memory metal mesh may comprise in whole or in part a non-braided structure.

### BRIEF DESCRIPTION OF THE DRAWINGS

FIG. 1 is a schematic illustration of a preferred embodiment of the occlusion device of the invention that is used for Patent Foramen Ovale (PFO) having component details including sealed potential fenestration (102), metallic braided mesh (105), connecting waist of regular PFO occluder (107), and radiopaque marker (108) (A. Front view of the occlusion device, B. Side view of the occlusion device).

FIG. 2 is a schematic illustration of a preferred embodiment of the occlusion device of the invention that is used for Atrial Septal Defect (ASD) having component details including sealed potential fenestration (102), metallic braided mesh (105), connecting waist of ASD occluder (112) and radiopaque marker (108) (A. Front view of the occlusion device, B. Side view of the occlusion device).

FIG. 3 (Panels A-D) is a schematic illustration of a preferred embodiment of the occlusion device of the invention having component details including left atrial disc (101), largest sized sealed potential fenestration (102), medium-sized second sealed potential fenestration (103), smallest sized third sealed potential fenestration (104), metallic braided mesh (105), right atrial disc (106), connecting waist of regular PFO occluder (107), the radiopaque marker (108), connecting hub (116) attached to the pusher cable (109), screw-hub (110), electrodes (113) and patch (111) material (A. Front view of the occlusion device, B. Side view of the occlusion device, C. Illustration showing two patch (111) layers on metallic braided mesh (105) and crosssections of the device, one angle view AA, D. Crossections of the device, another angle view BB).

FIG. 4 (Panel A-C) is a schematic illustration of a preferred embodiment of the occlusion device of the invention having component details including left atrial disc (101), largest sealed potential fenestration (102), medium-second sealed potential fenestration (103), smallest third sealed potential fenestration (104), metallic braided mesh (105), right atrial disc (106), connecting waist of ASD occluder (112), the radiopaque marker (108), connecting hub (116) attached to the pusher cable (109), screw-hub (110), electrodes (113) and patch (111) material (A. Front view of the occlusion device, B. Side view of the occlusion device, C. Illustration showing three patch (111) layers on metallic braided mesh (105).

FIG. 5 is a schematic illustration of a preferred embodiment of the occlusion device of the invention named as tunnel PFO occluder, having component details including right disc diameter (114), left disc diameter (115), connecting hub (116), connecting waist of tunnel PFO occluder (117).

FIG. 6 is a schematic illustration of a preferred embodiment of the occlusion device of the invention named as tunnel PFO occluder, having component details including electrodes (113) and connecting waist of tunnel PFO occluder (117).

FIG. 7 is a schematic illustration of a preferred embodiment of the occlusion device of the invention named as tunnel PFO occluder, having component details including half-circle atrial discs (118), electrodes (113), and connecting waist of tunnel PFO occluder (117).

FIG. 8 is a schematic illustration of a preferred embodiment of the occlusion device of the invention named as tunnel PFO occluder, having component details including straight anchoring parts with 90° (119) for better device stability and reduced risk of device embolization and connecting waist of tunnel PFO occluder (117).

FIG. 9 is a schematic illustration of a preferred embodiment of the occlusion device of the invention having component details including angulated anchoring parts with 45° (120) for better device stability and reduced risk of device embolisation and connecting waist of tunnel PFO occluder (117).

FIG. 10 (Panel A-C) is a schematic illustration of a preferred embodiment of the occlusion device of the invention with one sealed potential fenestration (102) having component details including left atrial disc (101), right atrial disc (106), connecting waist of regular PFO occluder (107), connecting hub (116), patch (111) material (A. Alpha (α) refers to angulation between the connecting waist of regular PFO occluder (107) to the left atrial disc (101) and right atrial disc (106), L refers to the length in between the left atrial disc (101) and right atrial disc (106), or length of the connecting waist of regular PFO occluder (107), B. W refers to the width of the connecting waist of regular PFO occluder (107), C.Detailed illustration showing different shapes of fenestrations as X, Y, and Z forms.)

FIG. 11 (Panel A and B) is a schematic illustration of a preferred embodiment of the occlusion device of the invention with two sealed potential fenestrations (102) having component details including connecting hub (116) and patch (111) material (A. Detailed illustration showing different shapes of fenestrations as X, Y and Z forms, B.Detailed illustration of patch (111) material).

FIG. 12 is a schematic illustration of a preferred embodiment of the occlusion device of the invention with three sealed potential fenestrations (102) with possible different shapes as X, Y, and Z forms and having component details including connecting hub (116) and patch (111) material.

FIG. 13 is a schematic illustration of a preferred embodiment of the occlusion device of the invention with four sealed potential fenestrations (102) with possible different shapes as X, Y, and Z forms and having component details including connecting hub (116) and patch (111) material.

### DESCRIPTION OF REFERENCES

101. left atrial disc

102. sealed potential fenestration

103. second sealed potential fenestration

104. third sealed potential fenestration

105. metallic braided mesh

106. right atrial disc

107. the connecting waist of regular PFO occluder

108. radiopaque marker

109. pusher cable

110. screw-hub

111. patch

112. the connecting waist of ASD occluder

113. electrodes

114. right disc diameter

115. left disc diameter

116. connecting hub

117. the connecting waist of tunnel PFO occluder

118. half circle atrial disc

119. Anchoring parts with 90°

120. Anchoring parts with 45°

AA: Crossections of the device drawings, one angle view.

BB: Crossections of the device drawings, another angle view.

α: angulation between the connecting waist of regular PFO occluder (107) to the left atrial disc (101) and right atrial disc (106).

L: length in between left atrial disc (101) and right atrial disc (106)

W: width of the connecting waist of the regular PFO occluder (107)

X, Y, and Z: refers to different forms of sealed potential fenestration (102) shapes

### DETAILED DESCRIPTION OF THE INVENTION

The present invention discloses a medical implantable interatrial septal defect occlusion device for percutaneous closure of interatrial septum defects such as Atrial Septal Defect (ASD) and Patent Foramen Ovale (PFO) by providing a closure at the defect area. There are three embodiments in the present invention. One embodiment provides an interatrial septal occluder for ASD. Another embodiment provides a regular interatrial septal occluder for PFO and the other embodiment provides another interatrial septal occluder for PFO, named as tunnel PFO occluder device.

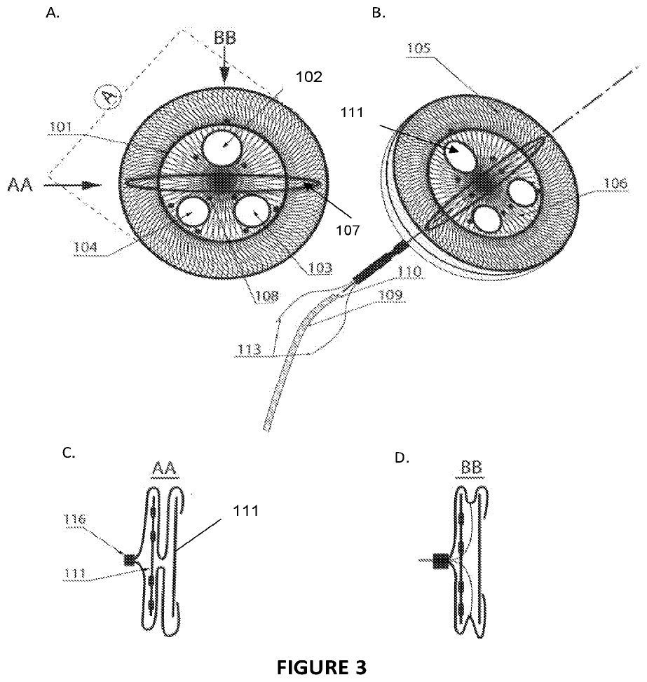

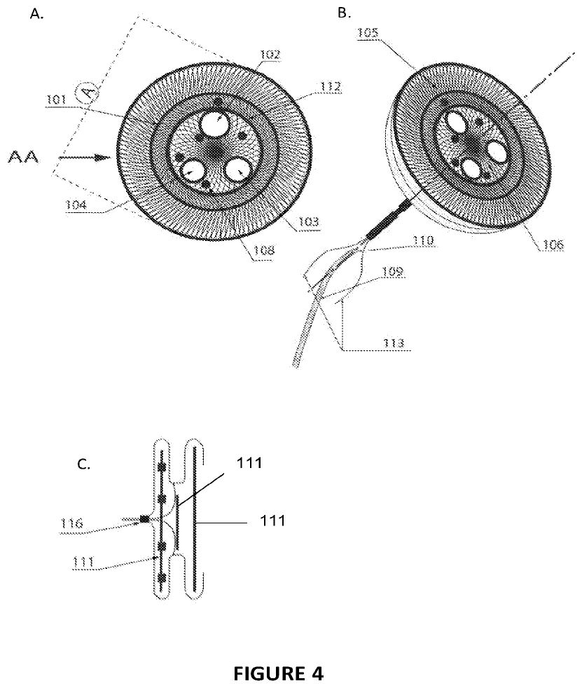

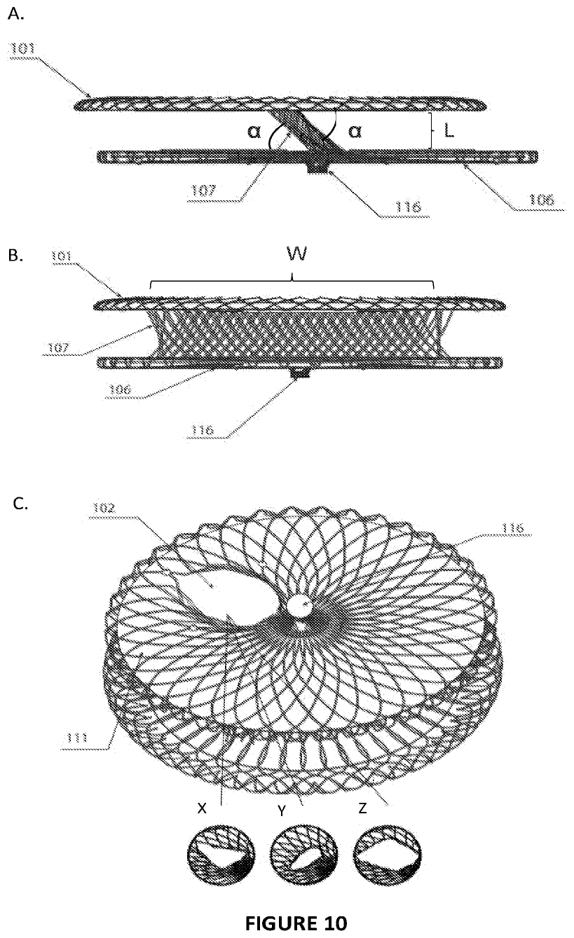

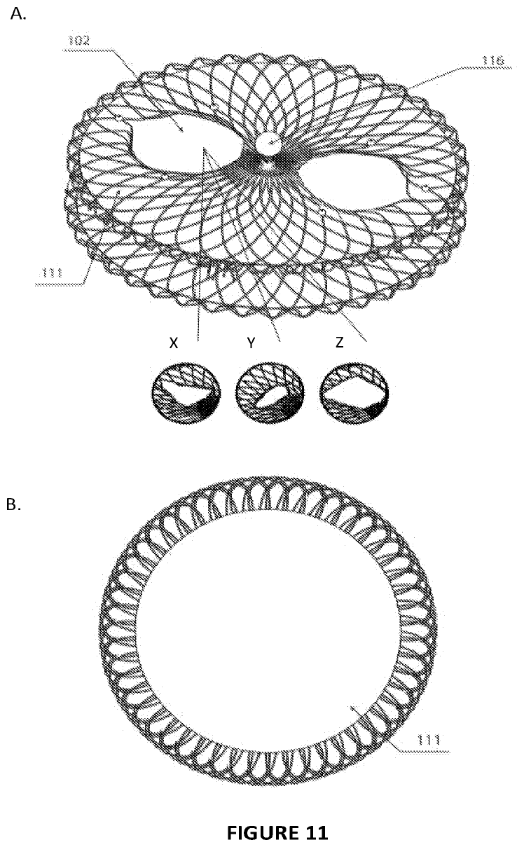

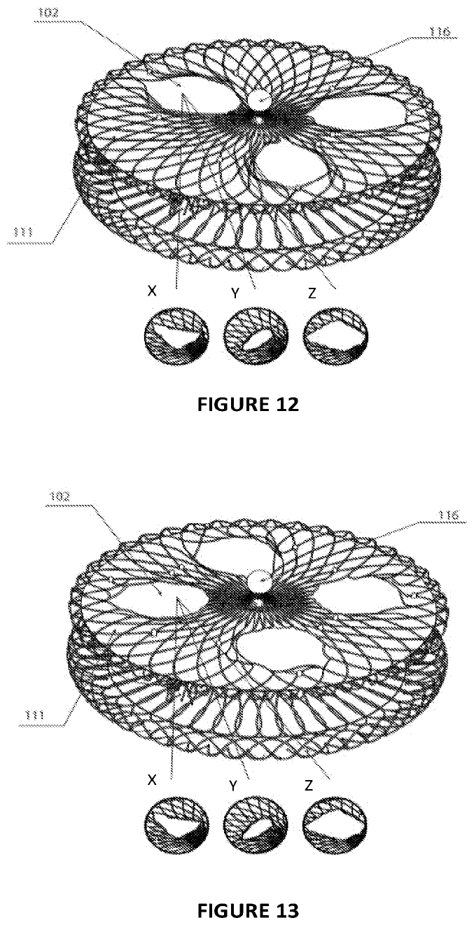

The subject matter of the occlusion device contains two discs made of metallic braided mesh (105). The metallic braided mesh (105) can be made of metal alloy which exhibits shape memory and superelasticity features. Said metal alloy can be nitinol or other metal alloys having shape memory and superelasticity features. In FIG. 3 and FIG. 4, the device contains two discs as left atrial disc (101) and right atrial disc (106). There is at least one sealed potential fenestration (102) located on metallic braided mesh (105) discs. There can be more than one sealed potential fenestration (102), as can be seen from FIG. 3, FIG. 4, FIG. 10, FIG. 11, FIG. 12, and FIG. 13. In one embodiment of the invention, there are three sealed potential fenestrations (102) as first, second, and third, sealed potential fenestrations. Sealed potential fenestration (102) which has the largest size among other fenestrations, second sealed potential fenestration (103) which has a medium size among other fenestrations, and a third sealed potential fenestration (104) which has the smallest size among other fenestrations. The advantage of having more than one fenestration is to meet the requirements of physicians when they need one fenestration for electrophysiology or/and intervention for structural heart diseases and at the same time need a fenestration for atrial flow regulation. For every intervention, physicians need different calibrations of fenestration. If a certain diameter of fenestration is needed for atrial flow regulation, the physicians will select the appropriate size among sealed potential fenestrations with different sizes to create the perforation. The sealed potential fenestrations (102, 103, 104) on the metallic braided mesh (105) surface provide later access to both sides of the atrium when an interventional trans-septal operation is needed to open the right atrial disc (106) on the right side of the interatrial septum.

On the right atrial disc (106) of the device, there is a connecting hub (116) attached to the pusher cable (109) which contains electrodes (113) to transfer energy like radio frequency (RF), heat, or similar for creating a fusion between the device surface and the tissue. The electrodes (113) are extensions of the energy cable inside the pusher cable (109), the connecting hub (116) is mounted on the surface of metallic braided mesh (105) with screw-hub (110).

The subject matter of the device has an angulation between the metallic braided mesh (105) discs (101,106) and connecting waist shown as alpha(α) in FIG. 10. The angle can be between 15°-90° (degree). Anatomically PFO opening or the tunnel has an angulation around 45 degrees in most of the cases. Therefore, the angulation between the discs (101,106) and connecting the waist of the regular PFO occluder (107) will create a better conformation to the PFO defect for better closure and less risk of the residual shunt.

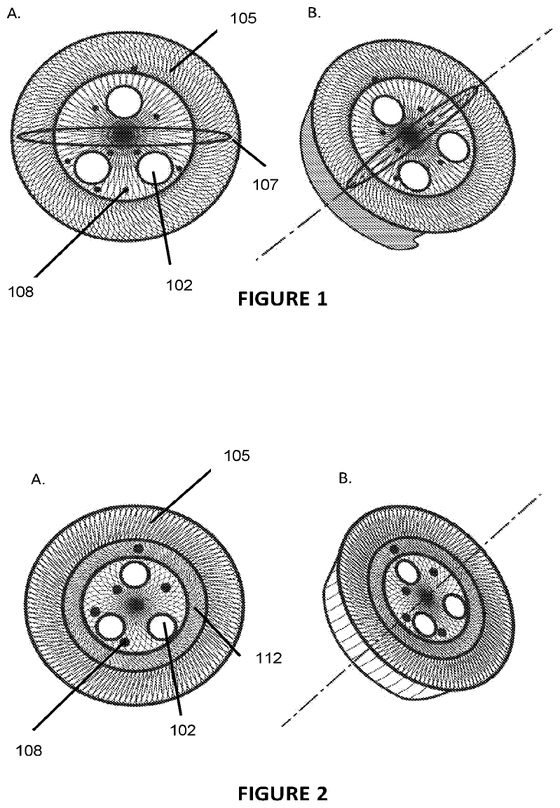

Sealed potential fenestrations (102) are sealed with the biocompatible polymeric patch (111) made from material like polytetrafluoroethylene (PTFE), polyethylene terephthalate (PET), synthetic polyester Dacron, polyurethane (PU), or bioabsorbable polymeric materials to provide sealing by creating a layer between both sides of the atria instantly and providing a surface for better endothelization. There is at least one layer of the patch (111) on the metallic braided mesh (105). Metallic braided mesh (105) is the frame of the device which is a uniform structure. After the production of metallic braided mesh (105), one or more patch (111) layer is sewed onto the metallic braided mesh (105). One layer of the patch (111) can be sewed on top of the disc and one patch can be sewed on the bottom of the disc. The sewing process is done by sutures made of PET or similar materials and a needle. After sewing is done, the suture is sealed by welding to secure the sutures. In one embodiment of the invention that is used for ASD, the occlusion device comprises three layers of the patch (111) on metallic braided mesh (105), wherein one of the layers is located on the left atrial disc (101), the other layer is located on the right atrial disc (106) and the other layer is located in the connecting waist of the ASD occluder (112) and to prevent flow in between atria. In another embodiment of the invention that is used for PFO, two layers of the patch (111) are located in the PFO occluder device as left disc layer and right disc layer to provide hemostatic sealing. FIGS. 1-4 and FIG. 10-13 show sealed potential fenestrations (102) and/or patch (111) on metallic braided mesh (105). Since sealed potential fenestrations (102) are sealed with the patch (111) and these two features are overlapped in structure, the references in figures also overlapped. Therefore, in some figures, these overlapped features are shown with 102 or 111 reference number.

In the present invention, there is at least one radiopaque marker (108) to indicate the sealed potential fenestrations' (102,103,104) locations since the potential fenestrations are sealed by patch (111) and physicians would not determine the location of potential fenestrations without these markers. Withthese radiopaque markers (108) located on patch (111) on the right atrial disc (106), physicians can visualize these pre-created sealed potential fenestrations (102,103,104) and access the left atrium side and perforate the patches (111) on sealed potential fenestrations (102, 103, 104) if intervention is needed. The radiopaque marker (108) can be visualized under fluorescence light.

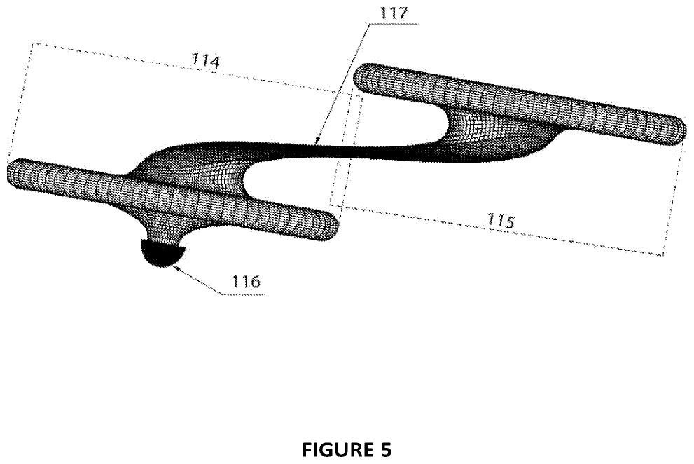

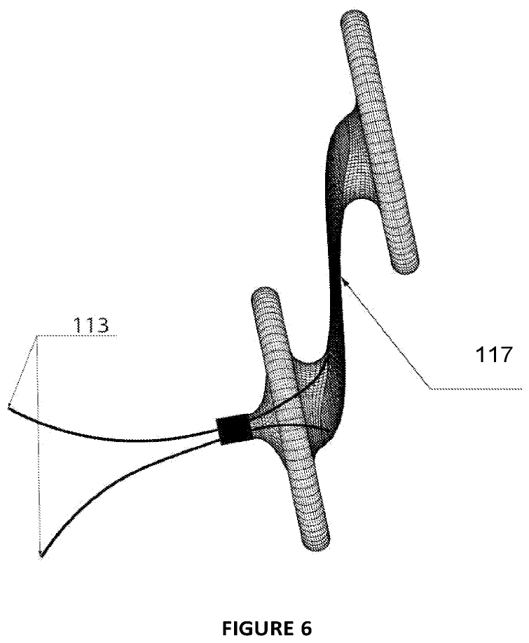

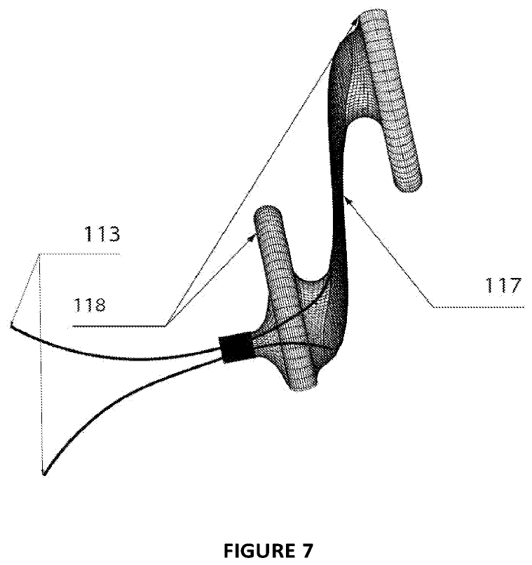

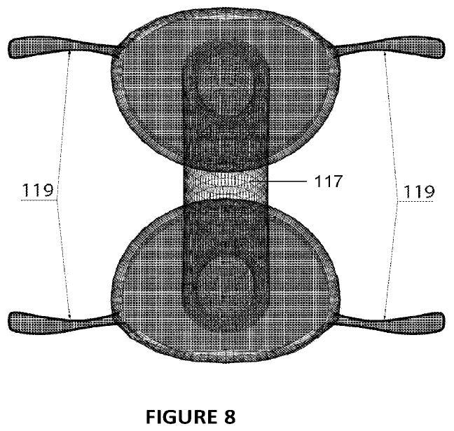

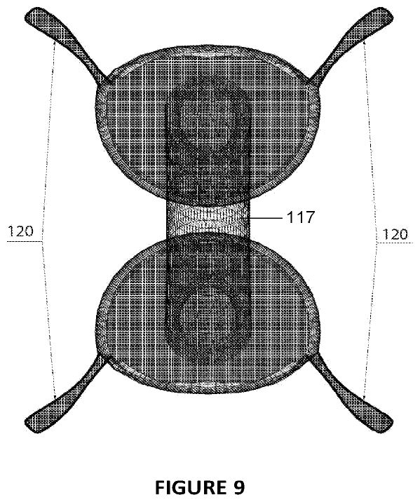

In the embodiment of the invention related to the tunnel PFO occluder, the device contains left atrial disc (101) and right atrial disc (106) in full circle atrial disc form as illustrated in FIG. 5 and FIG. 6; or half circle atrial disc (118) form on both sides, only to anchor the system in atrial septum as illustrated in FIG. 7. In tunnel PFO occluder embodiment, the PFO defect is closed by the internal connecting waist of the tunnel PFO occluder (117). The present invention provides a flat connection inside the interatrial defect with the connecting waist (107, 117) which is present in both of the PFO occluder embodiments as the connecting waist of the regular PFO occluder (107) shown in FIG. 1, FIG. 3, and FIG. 10; and connecting waist of the tunnel PFO occluder (117) shown in FIG. 5, FIG. 6, FIG. 7, FIG. 8, FIG. 9. The connecting waist of the tunnel PFO occluder (117) divides the PFO tunnel into two separate portions wherein the left side and right side connection is blocked, represented in FIG. 5, FIG. 6, FIG. 7. Even if there is a residual shunt with the disc closure, the blood flow right-to-left or left-to-right is blocked with the said connecting waist of the tunnel PFO (117). PFO occluders in the present invention (regular PFO occluder type and tunnel PFO occluder type) have flat connecting waists (107 and 117). In the embodiment of the invention related to ASD occluder, the connecting waist of the ASD occluder (112) is a cylindrically formed waist for ASD occluder in FIG. 4.

In the embodiment of tunnel PFO occluder, there are anchoring parts with 90° (119) or anchoring parts with 45° (120). The anchoring parts (119 or 120) do not attach to the septum. It is used for creating a clamping force for both sides of the structure. It can become from mesh or implemented to the mesh separately. It is made of metallic braided mesh (105) as the occluder device. In FIG. 8, a preferred embodiment of tunnel PFO occluder including straight anchoring parts with 90° (119) for better device stability and reduced risk of device embolisation. In FIG. 9, a preferred embodiment of tunnel PFO occluder including angulated anchoring parts with 45° (120) for better device stability and reduced risk of device embolisation.

An introducer system used in occlusion operation is placed to femoral vein and 0.035″ guidewire is advanced through the venous access site of the body passing from vena cava inferior, right atrium passing interatrial septum to the left atrium. A delivery (Mullin's) sheath is advanced over the guidewire until the tip of the catheter is placed to the desired location in the left atrium to give enough support to deliver the device to the defect location. The disclosed device is loaded to a loader and flushed with a saline solution to eliminate the risk of residual air bubbles which might cause air-embolization. The loader is connected to the delivery sheath with a male-female Luer locking mechanism. The subject matter of the occlusion device is connected to a pusher cable (109) with a screw-hub (110) system to mount and release the device at the desired location and time. The device is pushed by the help of a pusher cable (109) through the delivery system and the left atrial disc (101) of the device is opened in the left atrium and sometimes in pulmonary veins to give the left atrial disc (101) and pull back gently to the defect location and open the connecting waist (107, 112 or 117).

By pulling the pusher cable (109) gently, the physician tests the stability of the device at the implantation location and after being confided unscrew the pusher cable (109) to release the occlusion device. All the steps of the intervention are made with the guidance of fluoroscopy and/or Transesophageal Echocardiography (TEE) (2D or 3D). After the device is implanted in the desired location, the physician checks the stability of the device and controls with either contrast media flush or TEE color imaging for a residual shunt or any other silent ASD, PFO possibility. The delivery sheath, pusher cable (109), and all the system removed from the femoral vein access point of the patient, and the access point is closed.

In an embodiment of the invention, the diameter of the fenestration can be 3, 4, 5, 6, 8,10, or 12 mm. Sealed potential fenestration (102) can be in any geometrical shape and any diameter as can be seen from an example showing X, Y and Z forms in FIG. 10, FIG. 11, FIG. 12 and FIG. 13. In the present invention, right disc diameter (114) and left disc diameter (115) can be different according to embodiments. In FIG. 10, in an embodiment of the invention, said angulation (α) of the connecting waist of regular PFO occluder (107) can be in between 15°-90° (degrees); said length (L) in between the left atrial disc (101) and right atrial disc (106) can be in between 2-16 mm, said width (W) of the connecting waist of the regular PFO occluder (107) can be 3-16 mm.

What is claimed is:

1. A device for occluding an interatrial septal defect, said device comprising:
an expandable frame structure formed from a shape-memory metal mesh and having a left atrial disc, a right atrial disc, and a waist connecting the left atrial disc and the right atrial disc (106);
at least one fenestration located in the left atrial disc;
at least one fenestration located on the right atrial disc; and
a biocompatible polymeric patch on the left atrial disc to seal the at least one fenestration and on the right atrial disc to seal the at least one fenestration, wherin said biocompatible polymeric patch is configured to be perforated to allow access therethrough when needed.

2. A device according to claim 1, further comprising at least one radiopaque marker located on the left and/or the right biocompatible polymeric patch to indicate a location of one or more of the fenestrations.

3. A device according to claim 1, further a connecting hub configured to be attached to a pusher cable which contains electrodes to transfer energy to the device surface and septum tissue for fusion.

4. A device according to claim 1, wherin the waist is in a flat form in between the left atrial disc and the right atrial disc.

5. A device according to claim 1, wherin the waist is in a cylindrical form in between the left atrial disc and the right atrial disc.

6. A device according to claim 1, wherein said biocompatible polymeric patch comprises a material selected from the group consisting of polytetrafluoroethylene (PTFE), polyethylene terephthalate (PET), a polyester (Dacron®), apolyurethane (PU), or a bioabsorbable polymeric material.

7. A device according to claim 1, comprising a plurality of fenestrations located on at least one of the left atrial disc and the right atrial disc of the metallic braided mesh.

8. A device according to claim 7, wherein at least some of the plurality of fenestrations (102) have different sizes from each other.

9. A device according to claim 1, comprising one layer of the biocompatible polymeric patch on the left atrial disc and a separate layer of the biocompatible polymeric patch located on the right atrial disc to provide hemostatic sealing.

10. A device according to claim 9, comprising three layers of the biocompatible polymeric patch on metallic braided mesh, wherein a first layer is located on the left atrial disc, a second layer is located on the right atrial disc, and a third layer is located in the waist to provide hemostatic sealing.

11. A device according to claim 1, wherein the left atrial disc and right atrial disc are formed as full circles.

12. A device according to claim 1, wherein the left atrial disc and right atrial disc are formed as half circles.

13. A device according to claim 1, wherein the waist is configured to allow an angulation bwtween the left atrial disc and the right atrial disc in a range from 15° to 90°.

14. A device according to claim 2, wherein the radiopaque marker is located on the patch of the right atrial disc only.

15. A device according to claim 1, wherein said metallic braided mesh is made of a shape memory metal alloy which is superelastic.

16. A device according to claim 15, wherein said metal alloy comprises a nickel-titanium alloy.

17. A device according to claim 1, wherein the shape-memory metal mesh comprises a fully braided structure.

18. A device according to claim 1, wherein the shape-memory metal mesh comprises a partially braided structure.

19. A device according to claim 1, wherein the shape-memory metal mesh comprises a non-braided structure.
